(19) United States
(12) Patent Application Publication
(10) Pub. No.: US 2026/0000870 A1
(43) Pub. Date: Jan. 1, 2026

(54) ROBOTIC SURGICAL SYSTEM, APPARATUS, AND METHOD

(71) Applicant: VANDERBILT UNIVERSITY, Nashville, TN (US)

(72) Inventors: John E. Peters, Nashville, TN (US); Daniel Solomon Esser, Nashville, TN (US); Eric John Barth, Nashville, TN (US); Robert James Webster, Nashville, TN (US); Abby M. Grillo, Nashville, TN (US)

(21) Appl. No.: 19/253,706

(22) Filed: Jun. 27, 2025

Related U.S. Application Data
(60) Provisional application No. 63/665,089, filed on Jun. 27, 2024.

(51) Int. Cl. A61M 25/01; A61B 34/30

(52) U.S. Cl. CPC A61M 25/0155; A61M 25/0113; A61B 2034/301

(57) ABSTRACT

A flexible fluidic actuator for robotically imparting translational or rotational motion to a tube of a concentric tube manipulator, the flexible fluidic actuator includes a gripper that is actuatable in response to fluid pressure to grasp the tube, and de-actuatable in response to relieving fluid pressure to release the tube. The flexible fluidic actuator also includes one or more actuator bellows that are actuatable in response to fluid pressure to move the gripper to an actuated position, and de-actuatable in response to releasing fluid pressure to return the gripper to a de-actuated position. The gripper is configured to move to the actuated position in a first translational or rotational direction, and to move to the de-actuated position in a second translational or rotational direction, opposite the first translational or rotational direction. The one or more actuator bellows are arranged on an actuator frame and configured to support the gripper, and wherein the flexible fluidic actuator is configured so that the frame, actuator bellows, and gripper occupy the same axial length.

### RELATED APPLICATION

This application claims the benefit of U.S. Provisional Application Ser. No. 63/665,089, which was filed on Jun. 27, 2024, the entirety of which is hereby incorporated by reference in its entirety.

### GOVERNMENT FUNDING

This invention was made with government support under grant number R01 NS120518 awarded by the National Institutes for Health (NIH). The government has certain rights in the invention.

### TECHNICAL FIELD

This disclosure is related to robotic surgical interventions that take place in magnetically sensitive environments, such as within the bore of a magnetic resonance imaging (MRI) scanner. This disclosure is also related to concentric tube manipulators, especially needle-scale manipulators and to the actuation unit systems used to drive the manipulators, especially in the magnetically sensitive environment.

### BACKGROUND

Epilepsy affects more than 50 million people worldwide, afflicting patients with debilitating seizures. While antiepileptic drugs are available, 20-40% of patients remain medically refractory, leaving surgical intervention as the remaining option. In these surgical interventions, the focus of the seizure, the hippocampus, is removed or destroyed. The gold standard of surgical epilepsy interventions is Anterior Temporal Lobectomy (ATL). While ATL shows a 70% seizure freedom rate, 50-60% of qualified patients are not referred. ATL is a highly invasive approach and so it's understandable why many patients may choose not to pursue it. But it's not just patients, several studies have shown that physicians also seem to be deterred as they're under-referring patients.

Laser Interstitial Thermal Therapy (LITT) is a less invasive option that shows 54% seizure freedom rate. LITT consists of deploying a laser probe through a burr hole drilled in the back of the skull under the guidance of magnetic resonance imaging (MRI). This procedure aims to ablate as much of the hippocampus as possible to eliminate the seizures. The hippocampus, however, is a naturally curved structure, and this procedure is performed along a straight trajectory. This mismatch can ultimately limit the ablation coverage, which might explain why LITT has a lower seizure freedom rate. There exists a need for a more effective minimally invasive approach to using LITT to ablate the hippocampus. Concentric tube manipulators, with thwir small size and high curvature, offer a potential solution.

Magnetic resonance imaging (MRI) is a powerful tool in surgical workflows that is typically used for both pre-operative and post-operative diagnostics. With its superior soft tissue contrast and lack of dangerous radiation, it has grown increasingly popular over computed tomography (CT) for many image guided therapies. Interventional use of MRI has significant promise to provide real-time feedback during surgery, but is currently limited due to the challenges of performing surgery inside the MRI scanner, which offers a harsh environment as its intense magnetic field and limited workspace excludes the use of bulky, ferromagnetic components. When the cylindrical scanner bore is populated with a patient, very little room is left for a medical device. Furthermore, scanner image quality can suffer from the electromagnetic and ferromagnetic signature given off by surgical components. Creative actuation strategies are required to meet the demands of this environment.

### SUMMARY

A robotic surgical system provides a minimally invasive percutaneous approach for robotically delivered interventions. The approach implements a safe, compact, and accurate robotic direct drive actuation system for a concentric tube manipulator. The direct drive actuation system design presented here overcomes several problems commonly found in concentric tube robots and MRI-compatible devices.

First, there is the issue of tube buckling which arises from unsupported tube lengths, frictional interaction forces between tubes, and external loads on the tube tip. Typically, concentric tube actuation units operate on the principle of manipulating the tubes at their base and therefore leave large unsupported lengths of the tubes between the actuation unit and the working end. As a result, the unsupported tube is at a greater risk of buckling subject to tube friction and external loads such as tissue interaction forces. To overcome this issue, the direct drive actuation system directly implements an actuator unit that manipulates the tubes near or even at their tips. The actuator unit advances the tube through its center, without lead screws or intermediary structures, such as transmission tubes, effectively eliminating the unsupported section. This results in a drastically decreased risk of buckling as the tube is supported by the actuator directly against the target structure.

Second, there is the issue of torsional windup which, similarly, arises from unsupported tube lengths and the forces and torques associated with concentric tubes. As explained above, actuating the tube at their bases results in unsupported lengths towards the tube tip. As a result, the tube can windup or twist, yielding different angular displacements between the actuated base and working tip of the tube. Just like buckling, this can be catastrophic to the concentric tube robot's accuracy and operation. By directly rotating the tube at or near its tip, the unsupported length can be effectively eliminated minimizing the amount of torsional windup.

Of note here is the fact that buckling and torsional windup are common issues in the world of concentric tube robots where nitinol needles are the most common tube being actuated. Optical fibers, commonly used in medical applications, are even less stiff than these needles making them much more prone to buckling and windup. Their flexible nature demands constant support along their entire length making them an extremely strong use case for this direct drive actuation system due to the ability to grip and manipulate the fiber at the working end.

Finally, the use of a lead screw or intermediary transmission tube drastically increases the overall length of the actuation unit. For example, consider a single degree of freedom (DOF) stage consisting of the tube attached to a lead screw cartridge or a large diameter intermediary. The length of the lead screw or intermediary must be at least equal to or greater than the desired linear displacement of the tube, known as the stroke length. Consequently, the overall length is, at a minimum, twice the stroke length. For multiple stages, this length issue stacks becoming especially critical for space constrained applications, such as MRI-compatible robotics. The robotic surgical system disclosed herein eliminates the need for these components by directly manipulating the tubes and effectively halving the minimum, overall device length. Further length savings are achieved by integrating the secondary stage actuator with the encoded, sliding cartridge. We refer to this as the ride along architecture, whereby a secondary actuator and its corresponding tube (e.g., a fiber) can be pulled along as the first tube (e.g., a needle tube) is advanced. Typically, each actuator is rigidly fixed (stationary) separate from the tube cartridges increasing overall device length.

The system implements a fluid-driven (e.g., pneumatic) direct drive actuation unit for the concentric tube manipulator. The actuation unit implements a design that minimizes the overall device length while being compatible with MRI guided interventions. The actuation unit is configured to directly grip and manipulate the tubes, which yields the compact, short design, suitable for operation in the confined space of an MRI bore, which facilitates operation under MR visualization. By directly gripping the tubes, the actuation unit can generate torques and forces much closer to the point of insertion in the target, which helps reduce the risks that the tubes will undergo torsional windup or buckling.

Each tube of the concentric tube manipulator is actuated by a flexible fluidic actuator (FFA) assembly. Thus, for a two-tube configuration of the concentric tube manipulator, the actuation unit includes two FFA assemblies—one associated with each tube. Each FFA assembly is configured to impart two degrees-of-freedom (2 DOF) to its associated tube. Specifically, each FFA assembly is configured to impart rotational movement and translational movement to its associated tube. Accordingly, each FFA assembly includes a pair of flexible fluidic actuators: a translational FFA and a rotary FFA. The translational FFA includes a translational actuator and a tube gripper. The rotational FFA includes a rotational actuator and a tube gripper.

The tube grippers have a hollow center that allows the concentric tubes to extend through their respective FFAs. The grippers are selectively actuatable to grip the tube so that the FFA can impart its associated motion, i.e., translation or rotation, to the griped tube. The FFAs impart translational and rotational movement to the tubes through systematic, stepped actuation sequences under closed-loop control.

The FFAs have a nested configuration in which the gripper is supported by the actuator. Specifically, the gripper of the translational FFA is supported within a frame/housing of the translational actuator. The gripper of the rotational FFA is supported within a frame/housing of the rotary actuator. Because of this, the actuators and the grippers of each FFA occupy the same axial length, as opposed to being stacked against each other. As a result, the FFAs have a short, compact design, which offers several advantages.

First, implementing these FFA configurations allows the actuation unit, which includes one or more FFAs, to have a short, compact design. Advantageously, this allows the actuation unit to fit within the confines of the MRI tube so that interventions can be performed under MRI guidance. The short, compact design can allow the actuation to be oriented at steeper angles of attack, thus facilitating use in procedures that might otherwise be impossible with larger units, due to space limitations in the MRI tube.

Second, because the FFAs have short, nested configurations, they can be positioned close together in the actuation unit. This can allow for the flexible tubes of the concentric tube manipulator to be grasped directly for manipulation, as opposed to grasping transmission tubes. Torsional windup and lateral bending increase as the unsupported length of the tube increases. Traditional actuators position the contact points with the concentric tubes distant from the worksite. The resulting tube length between the actuation unit and the worksite necessitates the need for a transmission tube. The short, compact actuation unit disclosed herein allows the tubes to be grasped at locations close to the worksite so that transmission tubes, in some cases, can be avoided, and the flexible tubes can be grasped directly.

Finally, the grippers are configured to grasp needle-scale tubes which, by definition, have a 3 mm or less OD. The FFAs are therefore configured to translate/rotate needle-scale tubes. Given this scale, the actuation unit being so short and compact as to allow for direct grasping of a needle-scale flexible tube is highly advantageous. Needle-scale tubes can be difficult to machine, so applying a transmission tube can likewise be difficult. The FFAs of the actuation unit can avoid the need for a transmission tube altogether, in some instances.

According to one example configuration and implementation, the robotic surgical system can be used to deliver LITT under MRI guidance to treat epilepsy patients. To do this, the system uses a natural opening in the skull base—the foramen ovale—to access the brain. An outer cannula is docked in this opening and the concentric tube manipulator is deployed robotically into the brain. The concentric tube manipulator is configured to follow a trajectory that matches the natural curvature of the hippocampus. A laser probe extends out of the manipulator to ablate while pulling back along the hippocampal midline all under the guidance of intraoperative MRI, which helps maximize the amount of ablated hippocampal tissue.

A flexible fluidic actuator for robotically imparting translational or rotational motion to a tube of a concentric tube manipulator, the flexible fluidic actuator includes a gripper that is actuatable in response to fluid pressure to grasp the tube, and de-actuatable in response to relieving fluid pressure to release the tube. The flexible fluidic actuator also includes one or more actuator bellows that are actuatable in response to fluid pressure to move the gripper to an actuated position, and de-actuatable in response to releasing fluid pressure to return the gripper to a de-actuated position. The gripper is configured to move to the actuated position in a first translational or rotational direction, and to move to the de-actuated position in a second translational or rotational direction, opposite the first translational or rotational direction. The one or more actuator bellows are arranged on an actuator frame and configured to support the gripper, and wherein the flexible fluidic actuator is configured so that the frame, actuator bellows, and gripper occupy the same axial length.

According to one aspect, the flexible fluidic actuator can be configured to impart the translational or rotational motion to the tube in a first direction by sequentially actuating the gripper to grasp the tube, actuating the one or more actuator bellows to move the gripper to the actuated position thereby imparting translational or rotational movement to the grasped tube in the first direction, deactuating the gripper to release the tube, and de-actuating the one or more actuator bellows to return the gripper to the de-actuated position.

According to another aspect, the flexible fluidic actuator can be configured to impart the translational or rotational motion to the tube in a second direction, opposite the first direction by sequentially de-actuating the gripper, actuating the one or more actuator bellows to move the gripper to the actuated position, actuating the gripper to grasp the tube, de-actuating one or more actuator bellows to move the gripper to the de-actuated position thereby imparting translational or rotational movement to the grasped tube in the second direction.

According to another aspect, the gripper can include a pair of opposing gripper bellows supported on a gripper frame and configured to move a pair of opposing gripper jaws toward each other in the actuated condition of the gripper and to move the gripper jaws away from each other in the de-actuated state of the gripper.

According to another aspect, the flexible fluidic actuator can be a rotational flexible fluidic actuator configured to rotate the tube in opposite directions about the tube axis. The one or more actuator bellows can include a pair of actuator bellows configured to move opposite lateral ends of the gripper frame in opposite directions in order to impart rotational movement to the gripper.

According to another aspect, the flexible fluidic actuator can be a translational flexible fluidic actuator configured to translate the tube in opposite directions along the tube axis. The one or more actuator bellows can include a peripheral bellow that extends along a peripheral portion of the actuator frame and defines an open central window. The gripper can be connected to the peripheral bellow and positioned in the central window. The gripper can be configured to move axially with the peripheral bellow in opposite directions in order to impart translational movement to the gripper.

According to another aspect, the gripper can be a gripper tube supported within a gripper housing. The gripper tube can include a sidewall that defines a longitudinally extending central opening configured to receive the tube. The gripper tube sidewall can have a stellate corrugated configuration. The gripper housing can surround the gripper tube, defining a pressure chamber that extends circumferentially around the gripper tube between the gripper housing and the gripper tube. The pressure chamber can be pressurized with fluid in the actuated condition of the gripper, causing the gripper tube sidewall to deflect radially inward under fluid pressure into engagement with the tube to grasp the tube. Fluid pressure in the pressure chamber can be released in the de-actuated condition of the gripper, which causes the gripper tube sidewall to expand radially and release the tube.

According to another aspect, the corrugated configuration of the gripper tube sidewall can include axial corrugations and radial corrugations. The axial corrugations can extend the length of the gripper tube sidewall and can be spaced about the circumference of the gripper tube sidewall. The radial corrugations can extend about a central axis of the gripper tube and can be spaced along the length of the gripper tube.

According to another aspect, the gripper tube can be supported at each end by the gripper housing. The radial corrugations can be configured to cause the gripper tube sidewall to behave like a cantilevered beam where the accumulating pressure in the chamber yields max deflection towards the center of the gripper tube.

According to another aspect, the stellate configuration of the gripper tube can include a four-point stellate configuration defining four corrugations that produce four points of contact with the tube in the actuated condition of the gripper.

According to another aspect, the stellate configuration of the gripper tube can define peaks positioned proximally to the gripper housing and valleys positioned proximate to the tube.

According to another aspect, the flexible fluidic actuator can be a rotational flexible fluidic actuator configured to rotate the tube in opposite directions about the tube axis. A pair of actuator bellows can be configured to move opposite lateral ends of the gripper housing in opposite directions in order to impart rotational movement to the gripper.

According to another aspect, the flexible fluidic actuator can be a translational flexible fluidic actuator configured to translate the tube in opposite directions along the tube axis. A ring-shaped cylindrical bellow can define an open cavity configured to receive and support the gripper. The gripper can be configured to move axially with the cylindrical bellow in opposite directions in order to impart translational movement to the gripper.

According to another aspect, the gripper and the one or more actuator bellows can be configured for pneumatic actuation.

According to another aspect, an actuator unit for actuating inner and outer tubes of a concentric tube manipulator can include a first flexible fluidic actuator configured to robotically impart translational motion to the inner tube. A second flexible fluidic actuator can be configured to robotically impart rotational motion to the inner tube. A third flexible fluidic actuator can be configured to robotically impart translational motion to the outer tube. A fourth flexible fluidic actuator can be configured to robotically impart rotational motion to the outer tube.

According to another aspect, the actuator unit can include a frame configured to support the first, second, third, and fourth flexible fluidic actuators. The third and fourth flexile fluidic actuators can be fixed to the frame, and the first and second flexible fluidic actuators can be configured to move axially on the frame in response to translational movement of the outer tube with respect to the frame.

According to another aspect, the first and second flexible fluidic actuators can be supported by a first carriage including rollers configured to roll along the frame to allow the first and second flexible fluidic actuators to move axially on the frame.

According to another aspect, the actuator unit can include a second carriage including rollers configured to roll along the frame in response to translational movement of the inner tube with respect to the frame. The first carriage can include a linear encoder configured to measure the axial position of the outer tube, and a rotational encoder configured to measure the rotational position of the outer tube. The second carriage can include a linear encoder configured to measure the axial position of the inner tube, and a rotational encoder configured to measure the rotational position of the inner tube.

According to another aspect, all of the components of the actuator unit are constructed using an MRI compatible material.

According to another aspect, a surgical robotic system for performing a robotic intervention inside an MRI tube can include an actuator unit, a robotic parallelogram arm configured to support the actuator unit in the MRI tube and to robotically control the position of the actuator unit in the MRI tube, and a positioning platform to control the position of the robotic parallelogram arm and to fix the robotic parallelogram arm to a patient bed in the MRI tube.

According to another aspect, the robotic parallelogram arm can include a pair of flexible fluidic actuator each configured to control a degree of freedom of the parallelogram arm.

### DRAWINGS

FIG. 1 illustrates an example configuration of a robotic surgical system, apparatus, and method.

FIG. 2 is a perspective view of an apparatus that forms a portion of the robotic surgical system of FIG. 1 and is configured to perform the surgical method.

FIGS. 3-5 are perspective views of the apparatus with certain portions removed.

FIG. 6 is a perspective sectional view of the apparatus.

FIG. 7 is a perspective view illustrating first and second flexible fluidic actuator assemblies (FFA assemblies) of the apparatus.

FIG. 8 is a partially exploded perspective view of the first FFA assembly.

FIG. 9A is a perspective view of a first translational FFA, which is a component of the first FFA assembly.

FIG. 9B is an exploded perspective view of the first translational FFA.

FIG. 9C is a side sectional view of the first translational FFA.

FIG. 9D is a front view of the first translational FFA.

FIG. 10A is a perspective sectional view of a translational actuator component of the first translational FFA.

FIG. 10B is a side view of the translational actuator component of the first translational FFA.

FIG. 11A is a perspective view of a gripper component of the first translational FFA.

FIG. 11B is a perspective sectional view of the gripper component of the first translational FFA.

FIG. 11C is a front view of the gripper component of the first translational FFA.

FIG. 12A is a perspective view of a first rotational FFA, which is a component of the first FFA assembly.

FIG. 12B is a front view of the first rotational FFA.

FIG. 12C is an exploded perspective view of the first rotational FFA.

FIG. 13A is a perspective view of a rotational actuator component of the first rotational FFA.

FIG. 13B is a perspective sectional view of the rotational actuator component of the first rotational FFA.

FIG. 13C is a front view of the rotational actuator component of the first rotational FFA.

FIG. 14A is a perspective view of a gripper component of the first rotational FFA.

FIG. 14B is a front view of the gripper component of the first rotational FFA.

FIG. 14C is a side sectional view of the gripper component of the first rotational FFA.

FIG. 15A is a perspective view of the second FFA assembly.

FIG. 15B is a partially exploded perspective view of the second FFA assembly.

FIG. 16A is a perspective view of a second translational FFA, which is a component of the second FFA assembly.

FIG. 16B is an exploded perspective view of the second translational FFA.

FIG. 16C is a front view of the second translational FFA.

FIG. 16D is a side view of the second translational FFA.

FIG. 16E is a perspective sectional view of a gripper component of the second translational FFA.

FIG. 16F is a front sectional view of the gripper component of the second translational FFA.

FIG. 17A is a perspective view of a second rotational FFA, which is a component of the second FFA assembly.

FIG. 17B is an exploded perspective view of the second rotational FFA.

FIG. 17C is a perspective sectional view of the second rotational FFA.

FIG. 17D is a front view of the second rotational FFA.

FIG. 17E is a front sectional view of a gripper component of the second rotational FFA.

FIGS. 18A and 18B are sectional views illustrating portions of a gripper component.

FIGS. 19A and 19B are perspective views illustrating another configuration of an FFA assembly.

### DETAILED DESCRIPTION

### Robotic Surgical System 10

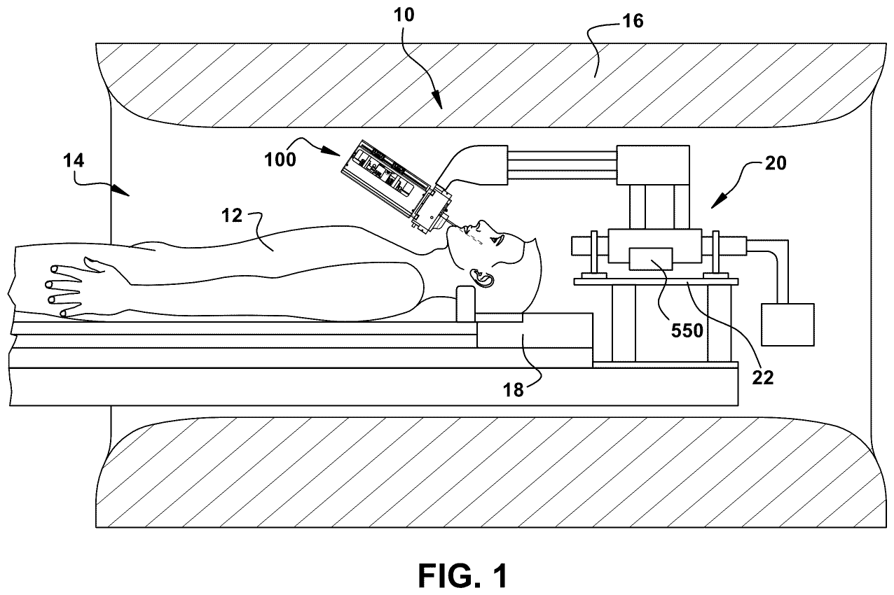

An example configuration of a robotic surgical system 10 is shown in FIG. 1. As shown in FIG. 1, the robotic surgical system 10 is used to perform surgical interventions on a patient 12 on a patient transfer board 18 in a scanner bore 14 of an MRI machine 16. The system 10 includes an apparatus in the form of an actuation unit 100 that is supported in the scanner bore 14 by a robotic parallelogram arm 20 mounted on a positioning platform 22.

### Actuation Unit 100

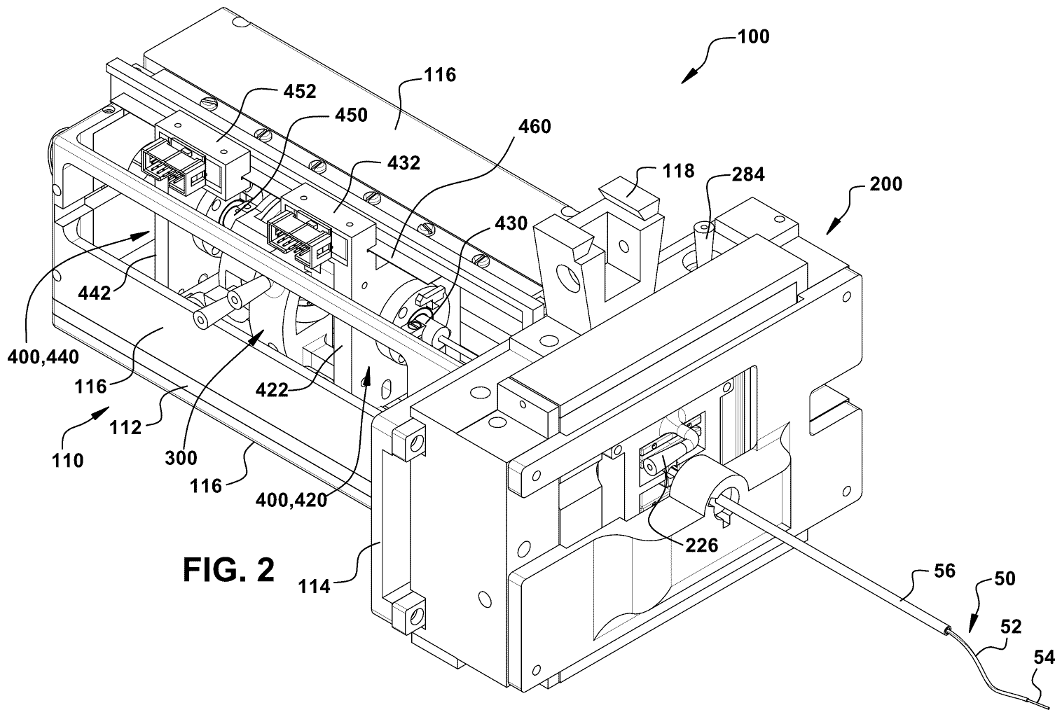

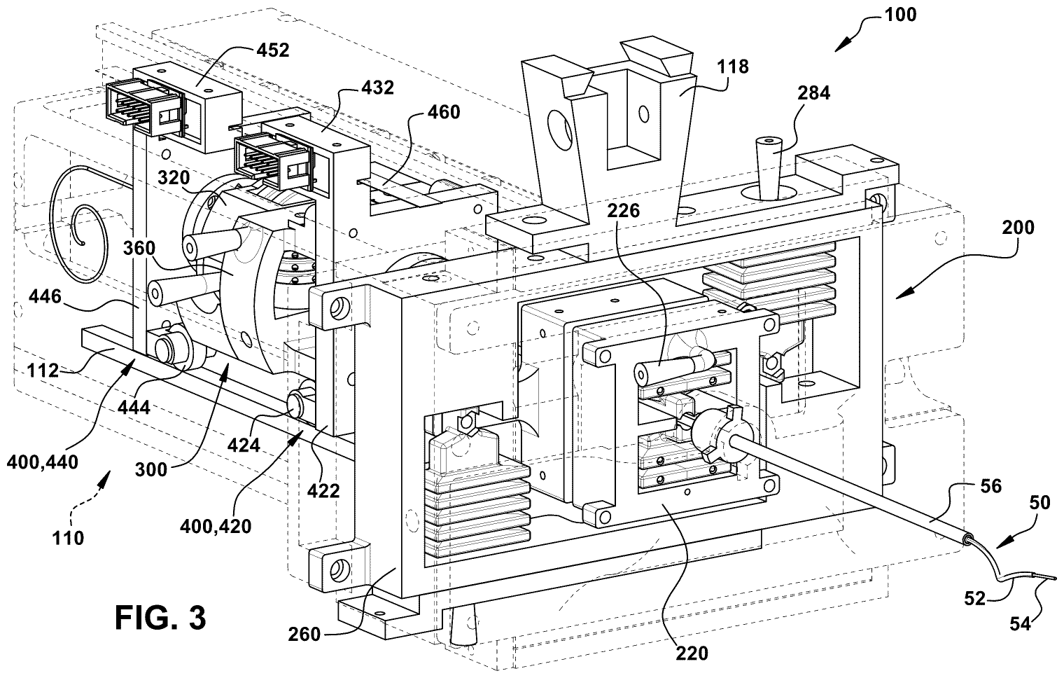

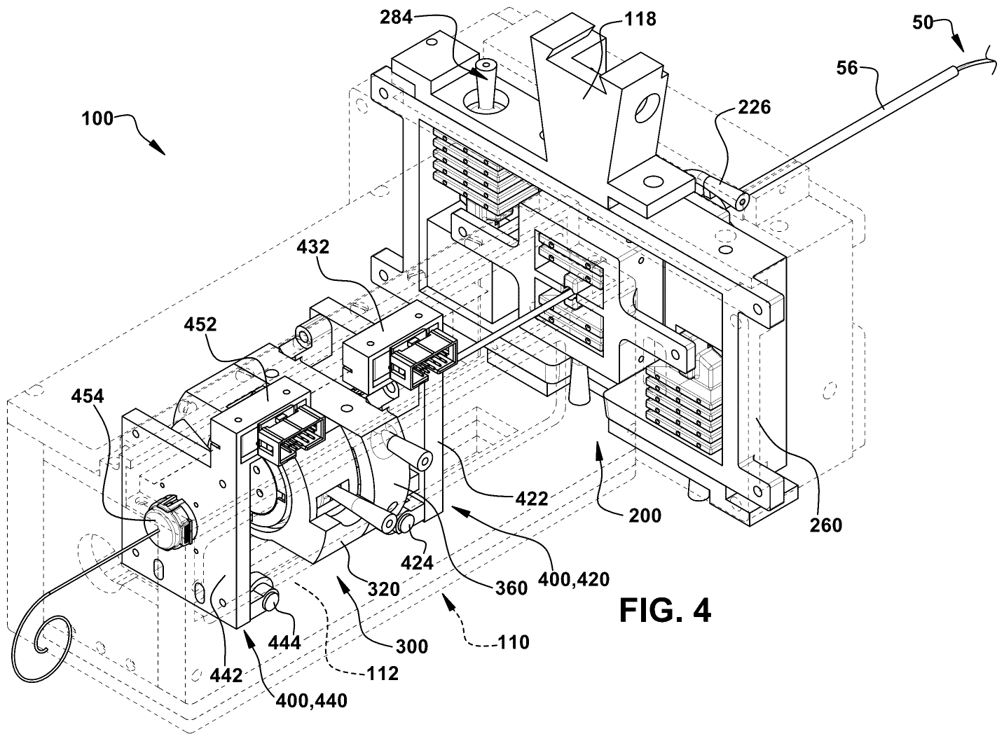

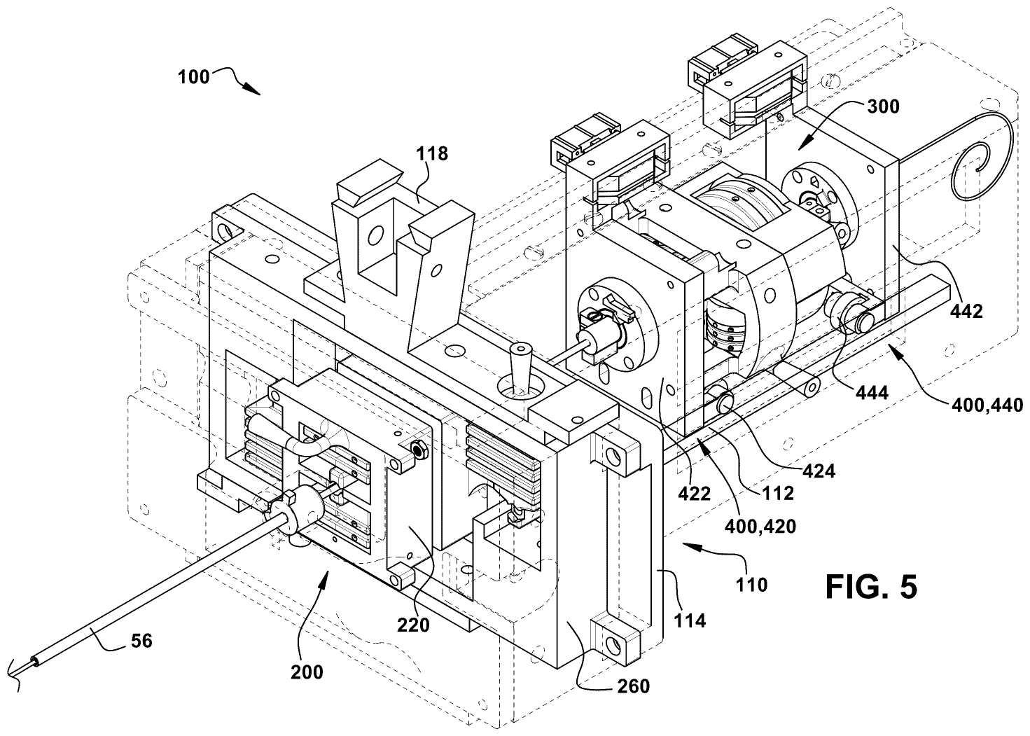

FIGS. 2-6 show various views of an example configuration of the actuation unit 100 in greater detail. The actuation unit 100 is configured to support and actuate a concentric tube manipulator 50. In the example configuration, the concentric tube manipulator includes an outer tubular member 52 and an inner member 54. As shown in FIGS. 2-6, the actuation unit 100 can also include a docking tube 56 that extends from the front of the unit. The concentric tube manipulator 50 is configured to be deployed through the docking tube 56. The docking tube 56 is, however, an optional component.

In one particular example configuration, the concentric tube manipulator 50 can be configured to perform a Laser Interstitial Thermal Therapy (LITT) surgical intervention, such as the therapy described hereinabove for treating epilepsy. In this example configuration, the outer tube 52 can be a pre-curved tube configured to be deployed through the foramen ovale and, from there, to follow a curved path along the midline of the hippocampus. The inner tube 54 can be a laser probe configured to exit the outer tube 52 and to be translated and rotated relative to the outer tube 52 in order to direct laser ablation energy toward the hippocampal tissue. In this example configuration, the docking tube 56 can be configured to deliver and “dock” the actuation unit 100 at the foramen ovale, and the concentric tube manipulator 50 can be configured to deployed from there.

The actuation unit 100 includes a frame 110 that supports and houses the various components of the actuation unit that are described herein. The frame 110 itself includes various components, including an L-frame 112 and a mounting plate 114 connected to the L-frame. The frame 110 also includes various covers 114 (e.g., top, bottom, and side) that protect the actuation unit components and also add rigidity and structural integrity to the actuation unit 100. The “frame 110,” as referred to herein, is meant to encompass these components and any other components that the actuation unit 100 might include for these purposes. The frame 110 can also include a merger plate 118 configured to connect the actuation unit 100 to the robotic parallelogram arm 20.

In the example configuration, the actuation unit 100 includes two flexible fluidic actuator (FFA) assemblies. The FFAs are fluidic in the sense that they are fluid (e.g., gas/air) driven, and flexible in the sense that they implement structures, such as bellows, that flex or deform in response to fluid pressure in order to impart actuation/movement.

A first FFA assembly 200 is mounted to the frame 110 (i.e., to the mounting plate 114) at the front end of the actuation unit and is configured to impart two degree-of-freedom (DOF) movement to the outer tube 52. A second FFA assembly 300 is mounted carriage assembly 420 and is configured to impart two DOF movement to the second tube 54 (e.g., the laser probe in the above-described LITT implementation).

### Concentric Tube Manipulators

In the example configuration, the concentric tube manipulator 50 utilizes a curved outer tube 52 to deliver an inner tube 54 in the form of a laser probe in order to perform the LITT intervention. The robotic surgical system 10 and the actuator unit 100 is not, however, limited to actuation of this particular concentric tube manipulator configuration. The system 10 and actuator unit 100 can be used to control the operation of any concentric tube manipulator configuration.

Concentric tube manipulators are small, needle-diameter, tentacle-like structures that include multiple concentric, pre-curved, elastic tubes. These elastic, curved tubes are typically made of a super elastic metal alloy such as a nickel-titanium alloy (“nitinol”) material. Individually or in combination, the tubes can be rotated about and/or translated along a common longitudinal axis of the concentric tubes. Through relative rotational movement, the rotational positions of the concentric tubes relative to each other can be controlled. Through relative translational movement, the concentric tubes can be retracted into one another and extended from one another.

As the pre-curved tubes interact with one another through relative translational and rotational movement, they cause one another to bend and twist, with the tubes collectively assuming a minimum energy conformation. The pre-curvature(s) of the tube(s) for a given manipulator can be selected to provide a desired workspace throughout which the tip can access through the relative rotational and/or translational movements of the tubes. The curved shape of the distal end of the manipulator is controlled via translation and rotation of each tube at a proximal location outside the patient, e.g., at its base inside a robot actuation unit where the tubes are connected to actuation unit elements.

The concentric tube manipulators are used to deliver instruments or tools, such as curettes, grippers, surgical lasers, grippers, retractors, scissors, imaging tips, cauterizing tips, ablation tips, morcellator, knives/scalpels, cameras, irrigation ports, suction ports, needles, probes, and tissue manipulators. The system 10 and actuator unit 100 can be used to robotically control the operation of a concentric tube manipulator having any combination of these characteristics and configurations.

### Materials and Construction

The actuation unit 100, including any and/or all of its components, can be constructed in a variety of manners using various materials. For instance, according to the example configuration described herein, the actuation unit 100 can be 3D printed. A 3D printed construction can be especially advantageous in situations, such as the LITT implementation, where MRI compatibility is important.

According to one example construction, the components of the actuation unit 100 can be constructed using material jetting, a printing method that utilizes additive manufacturing technology. This process works by jetting a liquid layer of resin droplets that cure and solidify under ultraviolet (UV) light. The main benefits of this technology are its extremely high printing resolution, the ability to use multiple different materials, and the ability to utilize a water-soluble support material. These benefits allow for printing components with very small corrugation patterns, desirable material properties, and hollow, internal vessels.

The material jetting method can allow the actuation unit 100 to be constructed using a blend of materials. In one example, a mixture of resin materials known as RGD515 Plus and RGD531, which are commercially available from Stratasys US of Eden Prairie, Minnesota (www.stratasys.com), can be used to construct the actuation unit 100 and its components. To do this, the actuation unit 100 can be printed on a Stratasys PolyJet J35 printer using this resin mixture, which produces the parts with a digital Acrylonitrile Butadiene Styrene (digital ABS) construction. The high tensile and flexural strength of digital ABS allow the components (e.g., bellows) to sustain high pressures while still undergoing relatively elastic elongation.

According to another example construction a selective laser sintering (SLS) method can be used. The SLS process involves where a bed of nylon 12 powder is laser sintered layer by layer to create a three dimensional structure. The un-sintered powder provides structural support to the component allowing for the printing of hollow pressure vessels. At the end of the printing process, the un-sintered powder drains out or is otherwise removed. SLS printing allows for high resolution printing, and the nylon material produces an incredibly strong, yet flexible elastic bellow. An example printer that can be implemented is a Formlabs Fuse 1 printer, available commercially from Formlabs Inc. of Somerville, Massachusetts.

### Carriage Assembly 400

The position of the first FFA assembly 200 is fixed on the actuation unit 100 and is therefore configured to move the outer tube 52 relative to the actuation unit and to the tip of any docking tube 56 or other structure through which it is delivered. The second FFA assembly 300 is mounted on a carriage assembly 400 configured for axial movement relative to the frame 110 and to the first FFA assembly 200. The carriage assembly 400 includes a first or front carriage 420 and a second or rear carriage 440. The front carriage 420 includes a frame 422 and connected rollers 424 configured to ride in slots 426 in the frame 110 (L-frame 112). Similarly, the rear carriage 440 includes a frame 442 and connected rollers 444 configured to ride in the slots 426.

The second FFA assembly 300 is mounted on the front carriage 420 and is movable with the front carriage axially along the frame 110. The front carriage 420 supports a coupling 430 that connects a rear end portion of the outer tube 52 to the front carriage. The coupling 430 is configured to allow the outer tube 52 to rotate freely, but also to move (push/pull) the front carriage 420 along the frame 110 along with the outer tube when it is translated relative to the frame. The front carriage 420 and the second FFA assembly 300 thus move axially along the frame 110 along with the outer tube 52 when translated axially by the first FFA assembly 200.

The front carriage 420 also supports a first linear encoder 432 configured to move with the front carriage along the frame 110. The first linear encoder 432 receives an encoder strip 460 that is fixed to the frame 110. As the first linear encoder 432 moves along the frame 110, the encoder strip passes 460 through, allowing for precise measurement of the position of the front carriage 420 relative to the frame 110. Because the front carriage 420 is connected to the outer tube 52 and configured to move along the frame 110 in response to translation of the outer tube, the first linear encoder 432 allows for precise measurement of the axial position of the outer tube.

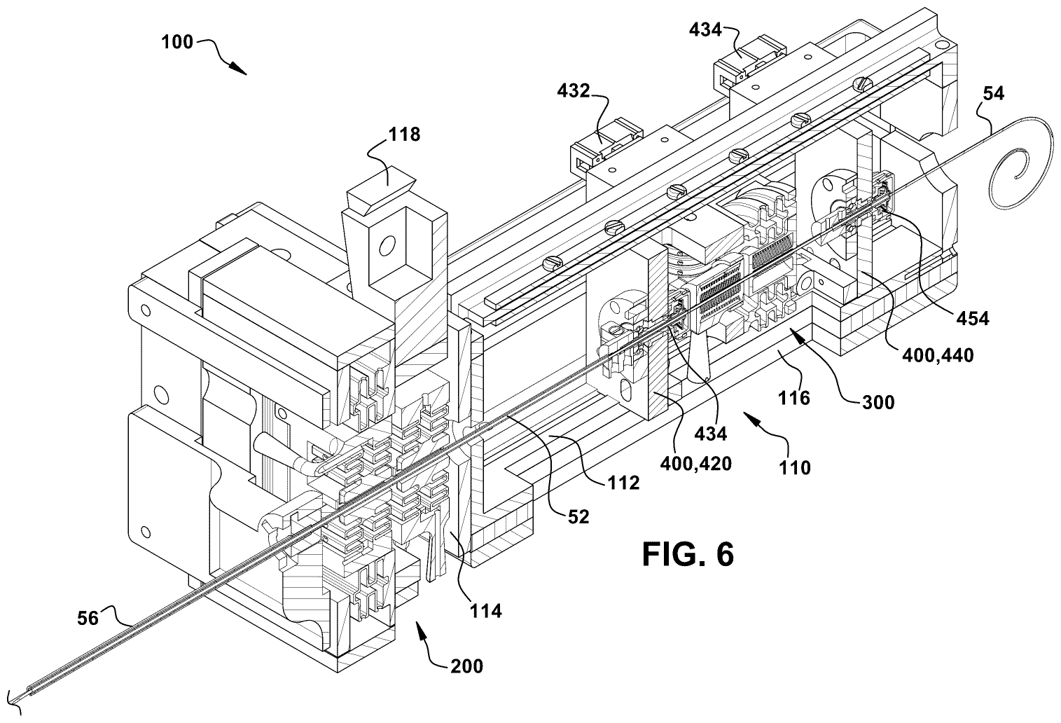

The front carriage 420 also supports a first rotary encoder 434 that is configured to measure the precise rotational position of the outer tube 52. The first rotary encoder 434 is shown only in FIG. 6 due to its being blocked from view in the other figures. The first rotary encoder 434 is, however, essentially identical to the second rotary encoder 454, which is shown clearly in FIG. 4 and described below.

The rear carriage 440 is movable axially along the frame 110. The rear carriage 440 supports a coupling 450 that connects the inner tube 54 of the concentric tube manipulator 50 to the rear carriage. The coupling 450 is configured to allow the inner tube 54 to rotate freely, but also to move (push/pull) the rear carriage 440 along the frame 110 along with the inner tube when it is translated relative to the frame.

The rear carriage 440 also supports a second linear encoder 452 configured to move with the rear carriage along the frame 110. The second linear encoder 452 also receives the encoder strip 460. As the second linear encoder 452 moves along the frame 110, the encoder strip passes 460 through, allowing for precise measurement of the position of the rear carriage 440 relative to the frame 110. Because the rear carriage 440 is connected to the inner tube 54 and configured to move along the frame 110 in response to translation of the inner tube, the second linear encoder 452 allows for precise measurement of the axial position of the inner tube. The rear carriage 440 also supports a second rotary encoder 454 that is configured to measure the precise rotational position of the inner tube 54. The second rotary encoder 454 is shown in FIG. 4.

### First and Second FFA Assemblies

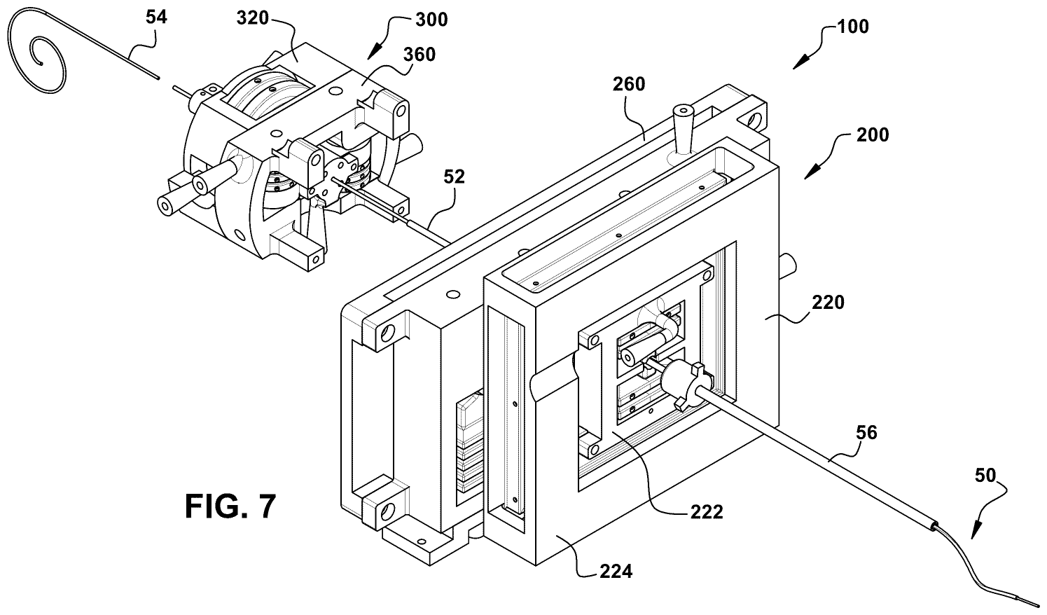

The first and second FFA assemblies 200, 300, along with the concentric tube manipulator 50, are shown in FIG. 7 with the remaining structure of the actuation unit 100 removed for clarity. The first FFA assembly 200 includes a first translational FFA 220 configured to impart translational movement to the outer tube 52, and a first rotational FFA 260 configured to impart rotational movement to the outer tube. The second FFA assembly 300 includes a second translational FFA 320 configured to impart translational movement to the inner tube 54, and a second rotational FFA 360 configured to impart rotational movement to the inner tube.

### First FFA Assembly 200

The first FFA assembly 200 is shown in FIG. 8. As shown in FIG. 8, the first translational FFA 220 and the first rotational FFA 260 can be separate units that are connectable to each other and/or to the frame 110 during assembly of the actuation unit 100.

### First Translational FFA 220

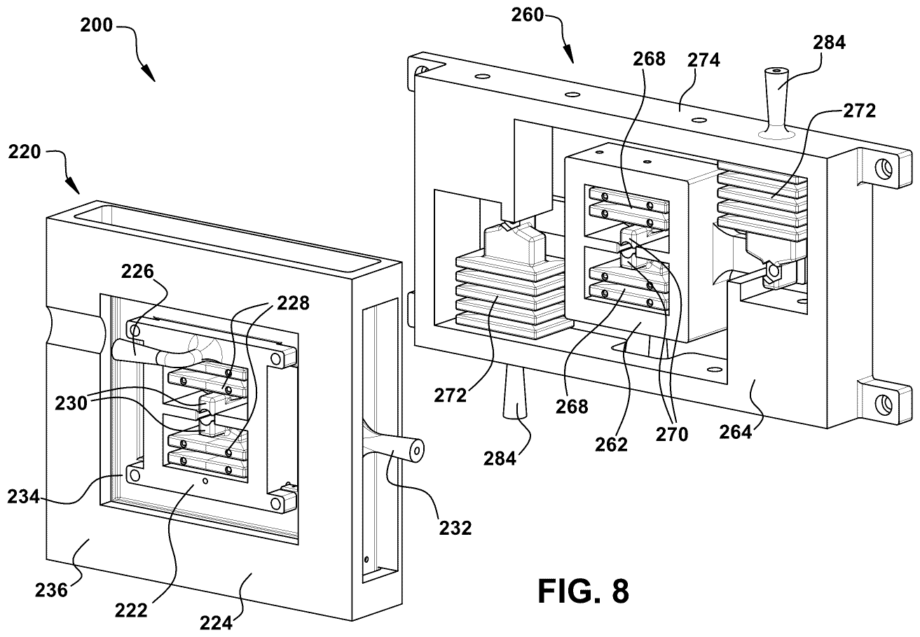

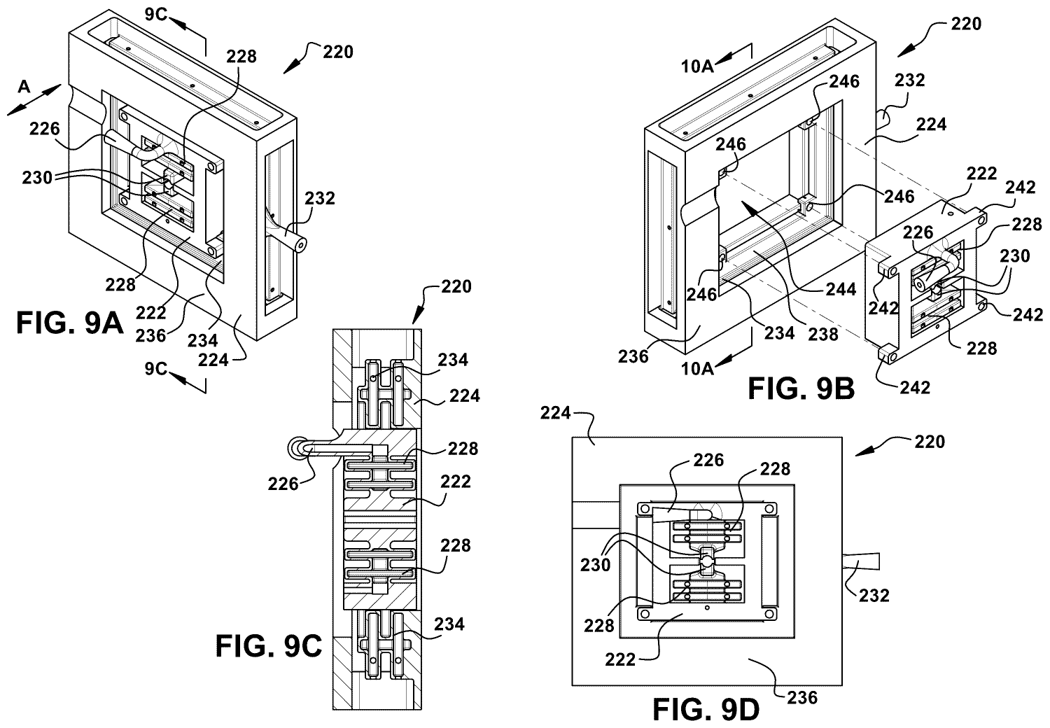

The first translational FFA 220 of the first FFA assembly 200 is also shown in FIGS. 9A-9D. The first translational FFA 220 includes a gripper 222 that is actuatable to grip the outer tube 52 and a translational actuator 224 that is actuatable to move the gripper axially. Thus, the first translational FFA 220 is configured to impart translational movement to the outer tube 52 when the translational actuator 224 is actuated while the gripper 222 is actuated to grip the outer tube.

### Gripper 222

The gripper 222 of the first translational FFA 220 is shown in FIGS. 8, 9A-9D, and 11A-11C, and can have a nylon SLS construction, as described above. The gripper 222 includes a pair of pneumatic bellows 228 supported on a rigid frame 240 in an opposing fashion. The bellows 228 of the gripper 222 have a generally rectangular/square cross-sectional configuration. The bellows 228 could, however, have alternative configurations, such as a rounded rectangular, round/circular, oblong/elliptical, etc. cross-sectional configuration.

Each bellows 228 supports a gripping jaw 230 at an end thereof. The opposing configuration of the bellows 228 is configured so that the gripping jaws 230 are themselves arranged in an opposing fashion. Each jaw 230 can have a semi-circular tube receiving surface that is configured to receive and engage the outer tube 52.

A fitting 226 defines an inlet for injecting a fluid, e.g., air for actuating the bellows 228. Under pressure of actuation air, the bellows 228 move the jaws 230 together to grip the outer tube 52. Venting the actuation air causes the bellows 228 to retract under their own resilience, thereby releasing the outer tube 52 from the grip of the jaws 230.

### Translational Actuator 224

The translational actuator 224 of the first translational FFA 220 is shown in FIGS. 8, 9A-9D, and 10A-10B, and can have a nylon SLS construction, as described above. The translational actuator 224 includes a singular bellows 234 that is supported on a rigid frame 236. The frame 236 has a rectangular configuration leaving an open central window 244 configured to receive the gripper 222. The bellows 234 is also rectangular and follows the rectangular geometry of the frame 236, and carries a frame 238 with an open central window 244 configured to receive the gripper 222 (see, especially, FIG. 9B).

While the bellows 234 is singular in the example configuration, the translational actuator 224 could have alternative configurations, such as a multi-bellow configuration where one or more bellows supports the frame 238 within the frame 236. In this instance, the bellows 228 could have alternative configurations, such as rectangular/square, rounded rectangular, round/circular, oblong/elliptical, etc. cross-sectional configurations. Additionally, the individual bellows also could be fluidly connected so as to produce uniform translational movement.

The frame 238 supported by the bellows 234 is configured to support the gripper 222 within the opening. The frame 240 of the gripper 222 includes tabs 242 that correspond to tabs 246 on the frame 238. The tabs 242, 246 are used to connect the gripper 222 to the frame 238 so that the gripper is supported by the bellows 234. The gripper 222, connected to the translational actuator 224 in this manner, is configured to move in response to actuation of the translational actuator.

The translational actuator 224 includes a fitting 232 that defines an inlet for injecting a fluid, such as air, for actuating the bellows 234. Under pressure of actuation air, the bellows 234 move the frame 238 and the connected gripper 222 axially back and forth in the direction of arrow A in FIG. 9A. Venting the actuation air causes the bellows 234 to retract under their own resilience, thereby returning the gripper 222 to a central/neutral position. The pressurization of the bellows 234 and release of pressure from the bellows is used to move the frame 238 and gripper 222 axially in the back and forth direction A. The translational actuator 224 can thus provide both forward and backward translational forces that are equal in magnitude, commensurate with the pressurization of the bellows 234 and the release of pressure from the bellows.

### First Rotational FFA 260

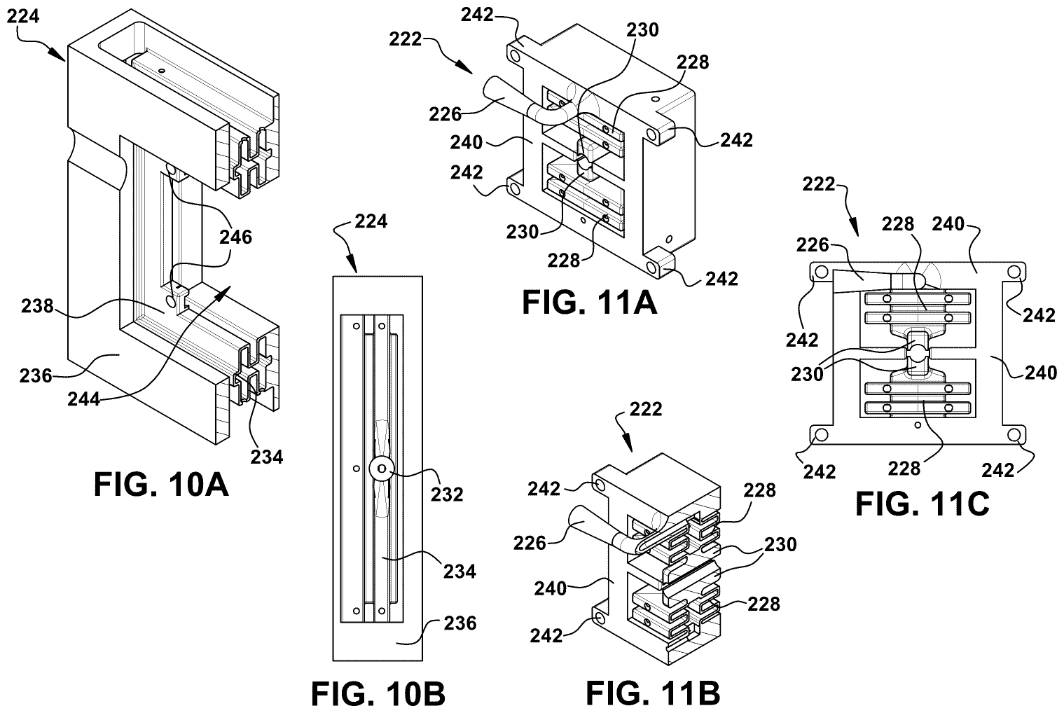

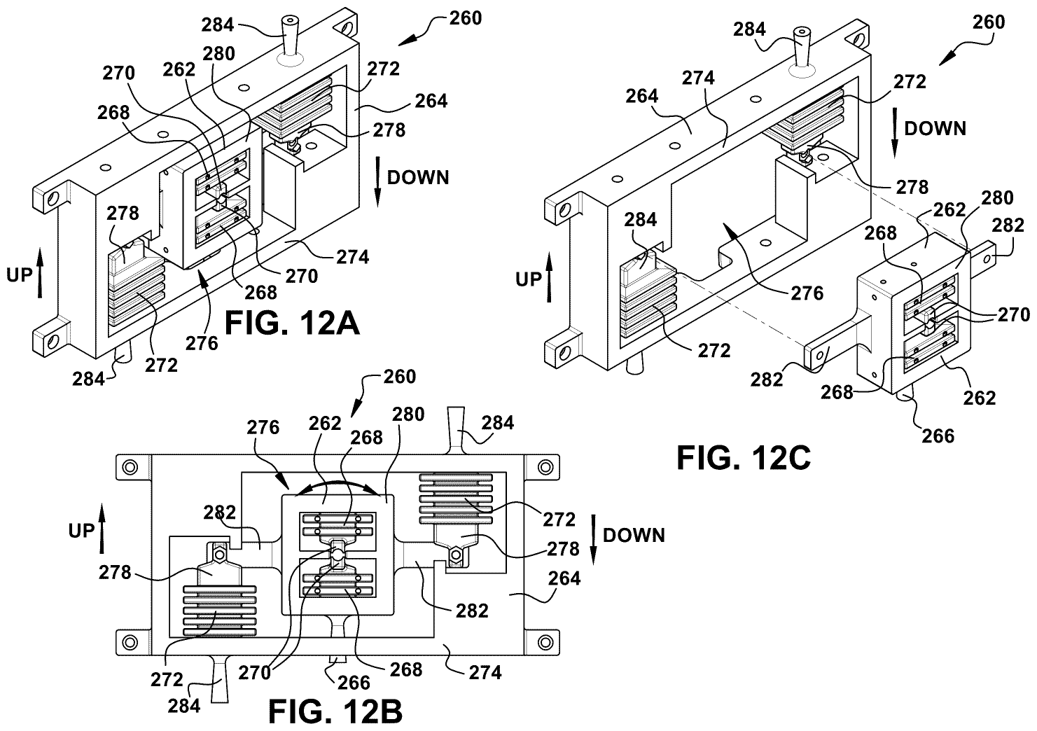

The first rotational FFA 260 of the first FFA assembly 200 is also shown in FIGS. 12A-12C. The first rotational FFA 260 includes a gripper 262 that is actuatable to grip the outer tube 52 and a rotational actuator 264 that is actuatable to rotate the gripper about the tube axis of the outer tube. Thus, the first rotational FFA 260 is configured to impart rotational movement to the outer tube 52 when the rotational actuator 224 is actuated while the gripper 222 is actuated to grip the outer tube.

### Gripper 262

The gripper 262 of the first rotational FFA 260 is shown in FIGS. 8, 12A-12C, and 14A-14C, and can have a nylon SLS construction, as described above. The gripper 262 includes a pair of pneumatic bellows 268 supported on a rigid frame 280 in an opposing fashion. The bellows 268 of the gripper 262 have a generally rectangular/square cross-sectional configuration. The bellows 268 could, however, have alternative configurations, such as a rounded rectangular, round/circular, oblong/elliptical, etc. cross-sectional configuration.

Each bellows 268 supports a gripping jaw 270 at an end thereof. The opposing configuration of the bellows 268 is configured so that the gripping jaws 270 are themselves arranged in an opposing fashion. The jaws 270 each have a semi-circular tube receiving surface that is configured to receive and engage the outer tube 52.

A fitting 266 defines an inlet for injecting a fluid, e.g., air, for actuating the bellows 268. Under pressure of actuation air, the bellows 268 move the jaws 270 together to grip the outer tube 52. Venting the actuation air causes the bellows 268 to retract under their own resilience, thereby releasing the outer tube 52 from the grip of the jaws 270.

The gripper 262 includes a pair of mounting arms 282 that extend laterally from the frame 280 in opposite directions. Each mounting arm 282 includes a mounting opening at an end portion thereof. The mounting arms 282 are configured to facilitate connecting the gripper 262 to the rotational actuator 264.

### Rotational Actuator 264

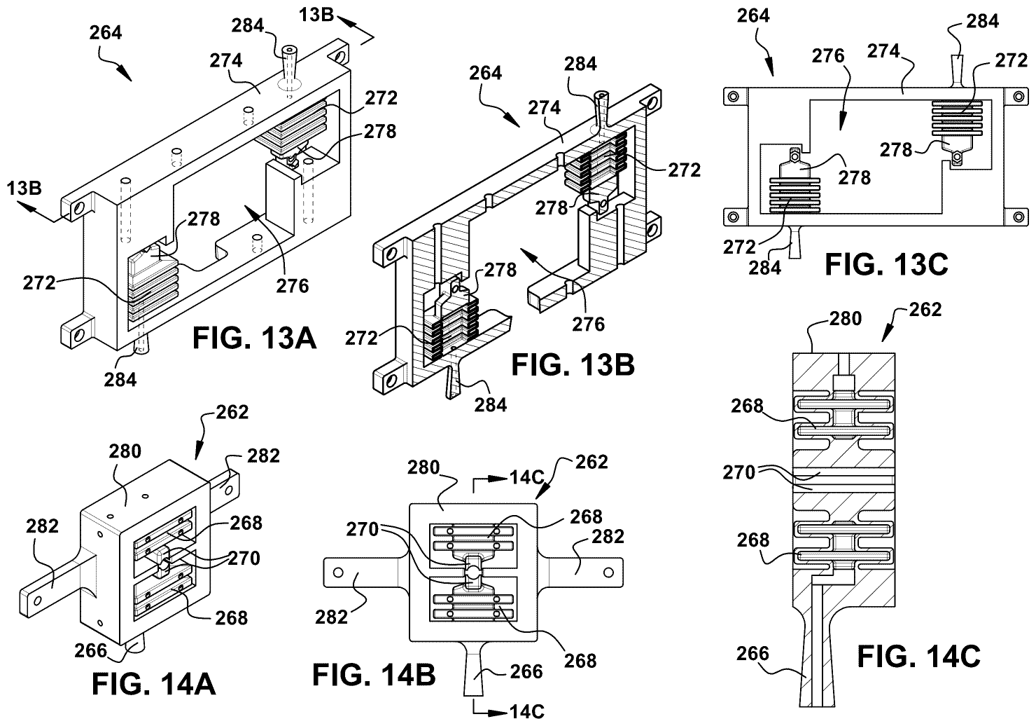

The rotational actuator 264 of the first rotational FFA 260 is shown in FIGS. 8, 12A-12C, and 13A-13C, and can have a nylon SLS construction, as described above. The rotational actuator 264 includes a pair of bellows 272 supported on a rigid frame 274. The frame 274 has a rectangular configuration and leaves open a central window 276 configured to receive the gripper 262. The bellows 272 are arranged in an opposing fashion on opposite sides of the frame 274. In the assembled condition of the first rotational FFA 260, the bellows 272 are positioned on opposite sides of the gripper 262, as shown in FIGS. 8 and 12A-12C. One of the bellows 272 is configured to be actuated in the UP direction, as indicated generally by the UP arrow in FIGS. 12A-12C, whereas the other bellows 272 is configured to be actuated in the DOWN direction, as indicated generally by the DOWN arrow in FIGS. 12A-12C.

Each bellows 272 supports a body 278 configured to move with the bellows as it is actuated. The body 278 is configured to cooperate with the mounting arms 282 of the gripper 262, particularly with the openings on the mounting arms, to connect the gripper frame 280 to the rotational actuator 264. The connection between the mounting arms 282 and the bellows bodies 278 is configured to allow the mounting arms to pivot relative to the bellows bodies. The connection can, for example, be in the form of a pin that extends through corresponding openings in the mounting arms 282 and the bellows bodies 278. The gripper 262 is thus connected to the rotational actuator 264 and configured to move in response to actuation of the rotational actuator.

Each bellows 272 of the rotational actuator 264 includes a fitting 284 that defines an inlet for injecting a fluid, e.g., air, for actuating the bellows. Under pressure of actuation air, each bellows 272 moves its associated body 278 in its respective UP/DOWN direction (see FIGS. 12A-12C). The mounting arms 282 of the gripper 262, being pivotally connected to the bellows bodies 278, moves in response to the actuation of its associated bellows. Actuated simultaneously, the bellows 272 impart rotation to the gripper 262.

Viewing FIGS. 12A-12C, positive pressure applied to the bellows 272 will cause one bellows (to the right as viewed in the figures) to actuate/expand in the DOWN direction, and the other bellows (to the left as viewed in the figures) to actuate/expand in the UP direction. Relieving or venting the pressure will cause the bellows 272 to actuate in opposite directions. As a result, for the configuration of FIGS. 12A-12C, pressurization of the bellows 272 will cause clockwise (CW) rotation of the gripper 262, and relieving/venting the pressure applied to the bellows will cause counterclockwise (CCW) rotation of the gripper. Venting or otherwise relieving the pressure from the bellows 272 will cause the rotational actuator 264, and the gripper 262 supported therein, to return to the neutral position shown, for example, in FIGS. 12A and 12B. This return motion is used to cause CCW rotation of the gripper 262. The rotational actuator 264 can thus provide both CW and CCW rotational forces that are equal in magnitude, commensurate with the pressurization of the bellows 272 and releasing of pressure from the bellows.

### Second FFA Assembly 300

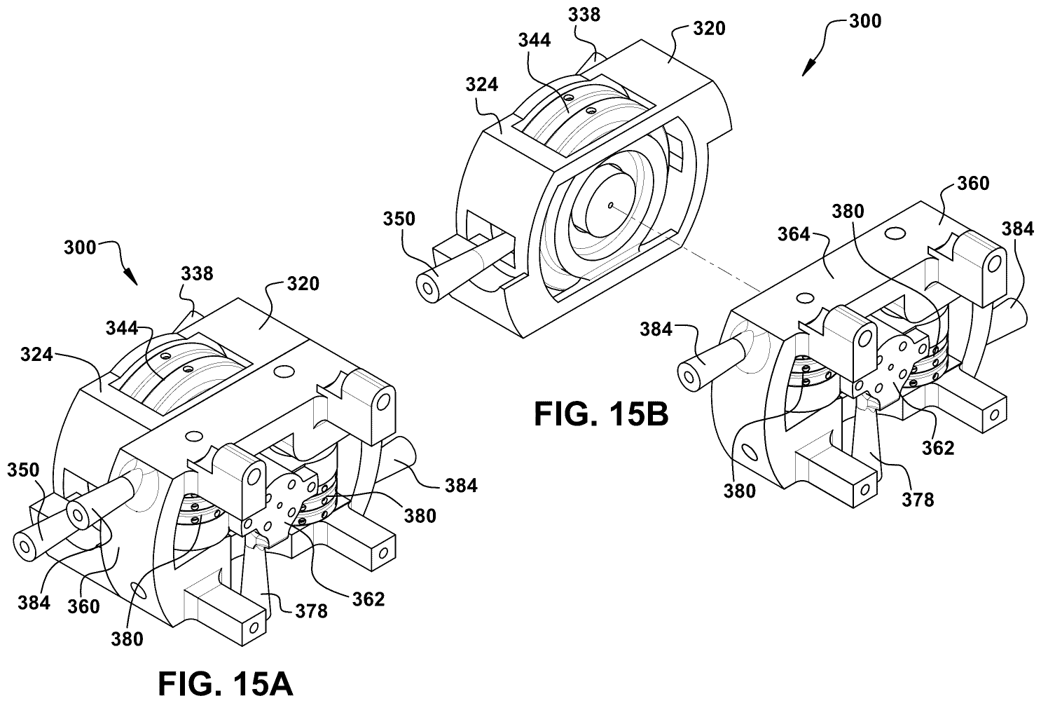

The second FFA assembly 300 is shown in FIGS. 15A-15B. As shown in FIGS. 15A-15B, the second translational FFA 320 and the second rotational FFA 360 can be separate units that are connectable to each other and/or to the frame 110 during assembly of the actuation unit 100.

### Second Translational FFA 320

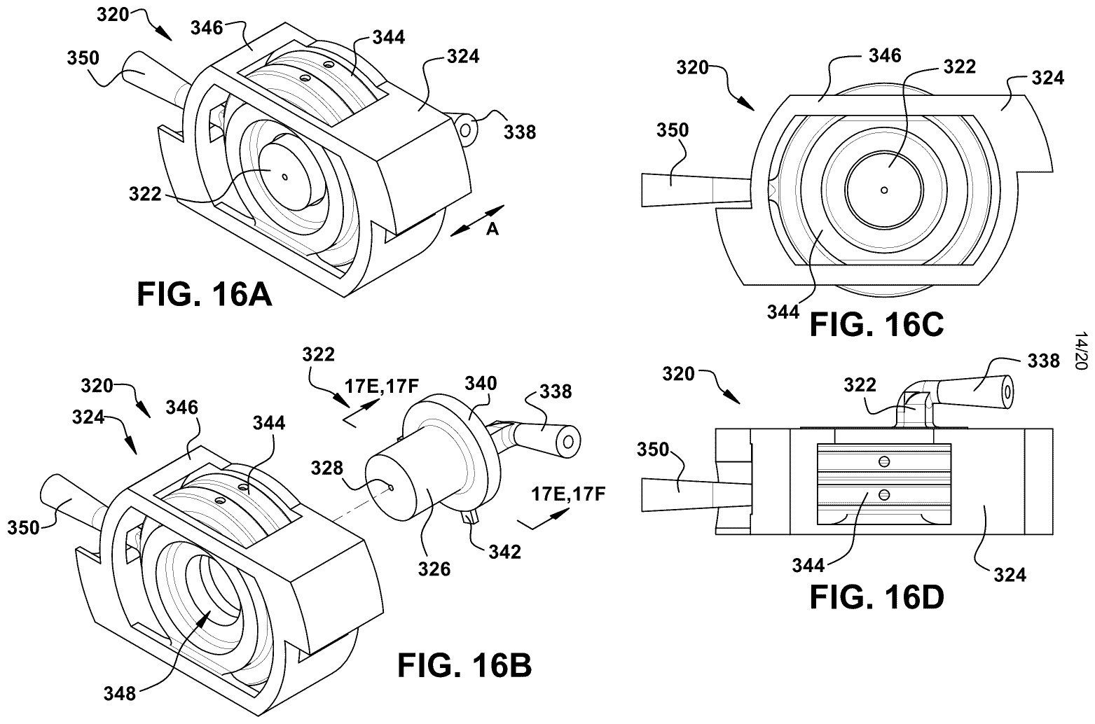

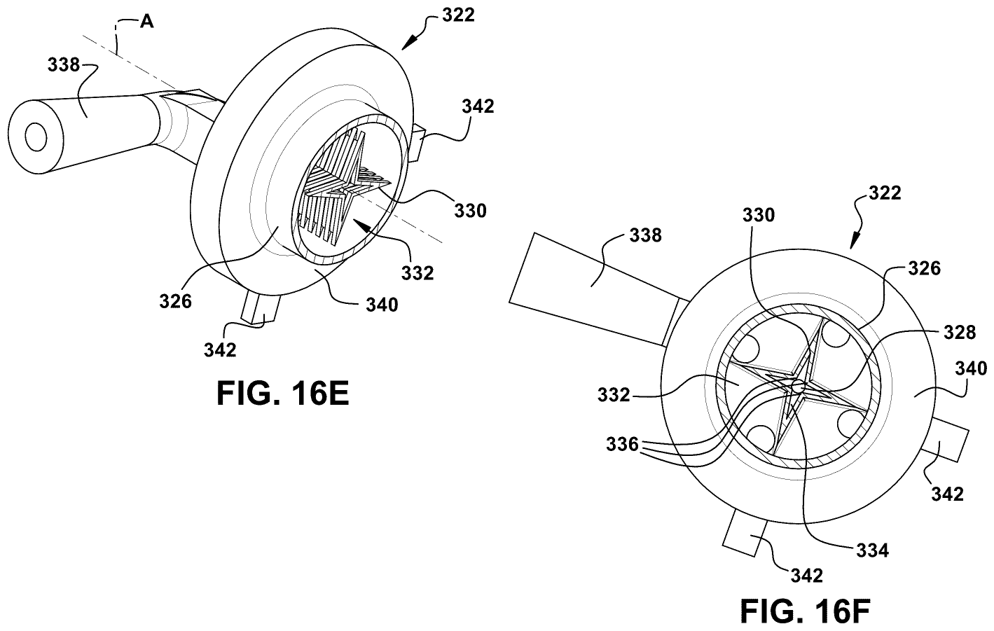

The second translational FFA 320 of the second FFA assembly 300 is also shown in FIGS. 16A-16F. The second translational FFA 320 includes a gripper 322 that is actuatable to grip the inner tube 54 and a translational actuator 324 that is actuatable to move the gripper axially. Thus, the second translational FFA 320 is configured to impart translational movement to the inner tube 54 when the translational actuator 324 is actuated while the gripper 322 is actuated to grip the outer tube.

### Gripper 322

The gripper 322 of the second translational FFA 320 is shown in FIGS. 16A-16F, and can have a printed digital ABS construction, as described above. The gripper 322 incudes a housing 326 with a central opening 328 configured to receive the inner tube 54 and allow the inner tube to pass therethrough. The gripper 322 includes a stellate (star-shaped), corrugated gripper tube 330 inside the housing 326. The opening 328 provides access to a gripping area 334 inside the gripper tube 330 through which the inner tube 54 passes.

A pressure chamber 332 inside the housing 326 but outside the gripper tube 330 can be pressurized with air to cause the gripper tube to deflect radially inward into engagement with the inner tube 54. When this occurs, gripping points 336 of the stellate gripper tube 330 engage and grip the inner tube 54.

A fitting 338 defines an inlet for injecting a fluid, e.g., air, for actuating the gripper tube 330. Under pressure of actuation air, the gripper tube 330 collapse and the gripping points 336 converge radially to grip the inner tube 54. Venting the actuation air causes the gripper tube 330 to retract under their own resilience, thereby releasing the inner tube 54 from the grip of the gripping points 336.

### Translational Actuator 324

The translational actuator 324 of the second translational FFA 320 is shown in FIGS. 16A-16D, and can have a nylon SLS construction, as described above. The translational actuator 324 includes a singular bellows 344 that is supported on a rigid frame 346, which has a generally rectangular form factor with rounded ends. The bellows 344 is mounted centrally in the frame 346 and has a ring-shaped configuration with an open cylindrical window 348 configured to receive the cylindrical housing 326 of the gripper 322.

The housing 326 of the gripper 322 includes a ring-shaped flange 340 from which a pair of tabs 342 extend. The tabs 342 correspond to slots on an end of the bellows 344 to which the gripper 322 is attached, for example, by an adhesive or a mechanical fastening. The gripper 322 is thus connected to the translational actuator 324 and configured to move in response to actuation of the translational actuator.

The translational actuator 324 includes a fitting 350 that defines an inlet for injecting a fluid, e.g., air, for actuating the bellows 344. Under pressure of actuation air, the bellows 344 move the gripper 322 axially back and forth in the direction of arrow A in FIG. 16A. Venting the actuation air causes the bellows 344 to retract under their own resilience, thereby returning the gripper 322 to a central/neutral position. The pressurization of the bellows 344 and release of pressure from the bellows is used to move the housing 326 and gripper 322 axially in the back and forth direction A (see FIG. 16A). The translational actuator 324 can thus provide both forward and backward translational forces that are equal in magnitude, commensurate with the pressurization of the bellows 344 and the release of pressure from the bellows.

### Second Rotational FFA 360

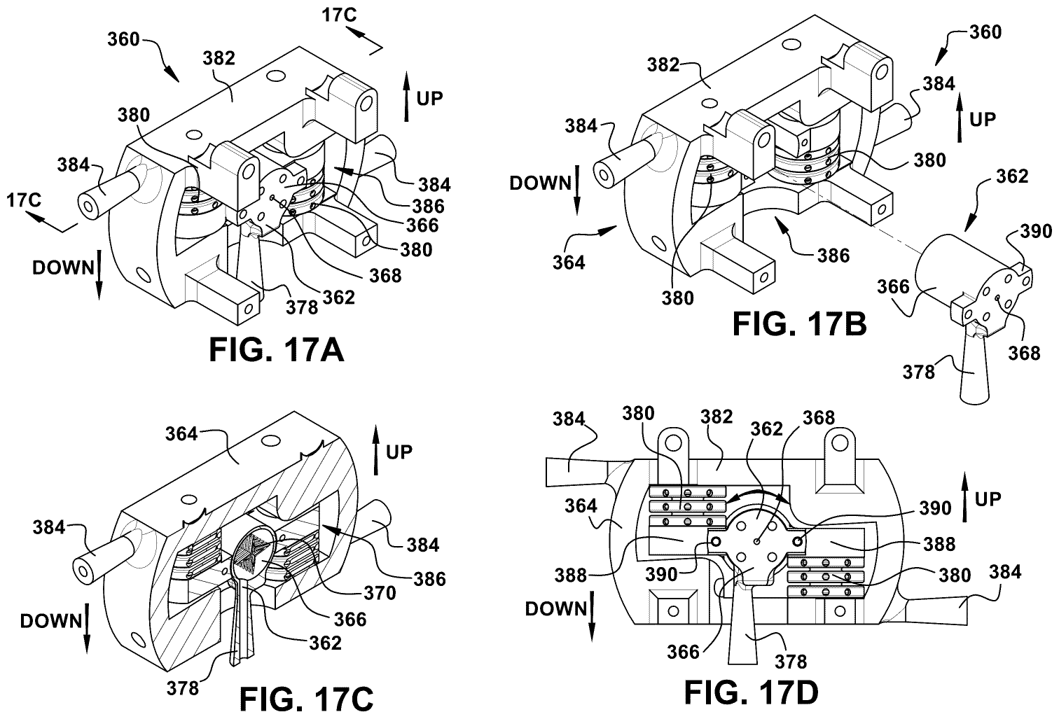

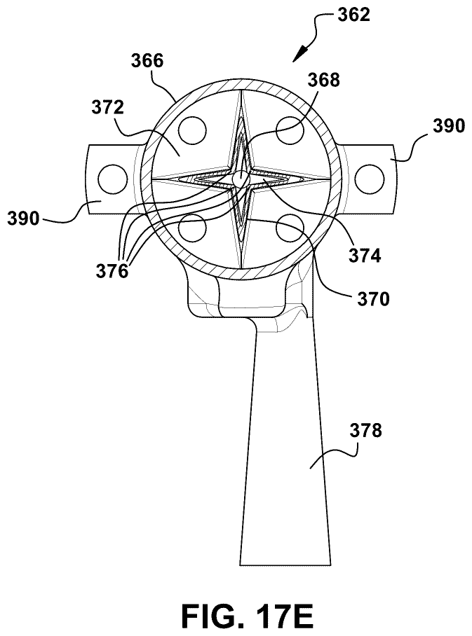

The second rotational FFA 360 of the second FFA assembly 300 is also shown in FIGS. 17A-17E. The second rotational FFA 360 includes a gripper 362 that is actuatable to grip the outer tube 52 and a rotational actuator 364 that is actuatable to rotate the gripper about the tube axis of the outer tube. Thus, the second rotational FFA 360 is configured to impart rotational movement to the outer tube 52 when the rotational actuator 364 is actuated while the gripper 362 is gripping the outer tube.

### Gripper 362

The gripper 362 of the second rotational FFA 360 is shown in FIGS. 17A-17E, and can have a printed digital ABS construction, as described above. The gripper 362 has a configuration that is very similar to that of the gripper 322 of the second translational FFA 320. The gripper 362 incudes a housing 366 with a central opening 368 configured to receive the inner tube 54 and allow the inner tube to pass therethrough. The gripper 362 includes a stellate gripper tube 370 inside the housing 366. The opening 368 provides access to a gripping area 374 inside the gripper tube 370 through which the inner tube 54 passes (see, especially, FIG. 17E).

A pressure chamber 372 inside the housing 366 but outside the gripper tube 370 can be pressurized with air to cause the gripper tube to contract radially inward into engagement with the inner tube 54. When this occurs, gripping points 376 of the stellate gripper tube 370 engage and grip the inner tube 54.

A fitting 378 defines an inlet for injecting a fluid, e.g., air for actuating the gripper tube 370. Under pressure of actuation air, the gripper tube 370 collapse and the gripping points 376 converge radially to grip the inner tube 54. Venting the actuation air causes the gripper tube 370 to retract under their own resilience, thereby releasing the inner tube 54 from the grip of the gripping points 376.

### Rotational Actuator 364

The rotational actuator 364 of the second rotational FFA 360 is shown in FIGS. 17A-17D, and can have a nylon SLS construction, as described above. The rotational actuator 364 includes a pair of bellows 380 supported on a rigid frame 382. The frame 382 has a form factor that is substantially similar to that of the translational actuator 320 so that the two substantially match each other when assembled together.

The frame 382 leaves open a central window 386 configured to receive the gripper 362. The bellows 380 are arranged in an opposing fashion on opposite sides of the frame 382. In the assembled condition of the second rotational FFA 360, the bellows 380 are positioned on opposite sides of the gripper 362, as shown in FIGS. 17A-17D. One of the bellows 380 is configured to be actuated in the UP direction, as indicated generally by the UP arrow in FIGS. 17A-17D; whereas the other bellows 372 is configured to be actuated in the DOWN direction, as indicated generally by the DOWN arrow in FIGS. 17A-17D.

Each bellows 380 supports a body 388 configured to move with the bellows as it is actuated. The body 388 is configured to cooperate with mounting arms 390 of the gripper 362, particularly with the openings on the mounting arms, to connect the gripper housing 366 to the rotational actuator 364. The connection between the mounting arms 390 and the bellows bodies 388 is configured to allow the mounting arms to pivot relative to the bellows bodies. The connection can, for example, be a in the form of a pin that extends through corresponding openings in the mounting arms 390 and the bellows bodies 388. The gripper 362 is thus connected to the rotational actuator 364 and configured to move in response to actuation of the rotational actuator.

Each bellows 380 of the rotational actuator 364 includes a fitting 384 that defines an inlet for injecting air for actuating the bellows. Under pressure of actuation air, each bellows 380 moves its associated body 388 in its respective UP/DOWN direction (see FIGS. 17A-17D). The mounting arms 390 of the gripper 362, being pivotally connected to the bellows bodies 388, moves in response to bellows actuation. Actuated simultaneously, the bellows 380 impart rotation to the gripper 362.

Viewing FIGS. 17A-17D, positive pressure applied to the bellows 380 will cause one bellows (to the left as viewed in the figures) to actuate/expand in the DOWN direction, and the other bellows (to the right as viewed in the figures) to actuate/expand in the UP direction. As a result, positive pressure applied to the bellows 380 will cause counterclockwise (CCW) rotation of the gripper 362. Venting or otherwise relieving the pressure from the bellows 380 will cause the rotational actuator 364 and the gripper 362 supported therein to return to the neutral position shown, for example, in FIGS. 17A and 17D. This return motion is used to cause clockwise (CW) rotation of the gripper 362. The rotational actuator 364 can thus provide both CW and CCW rotational forces that are equal in magnitude, commensurate with the pressurization of the bellows 380 and releasing of pressure from the bellows.

### Stellate Gripper Tube

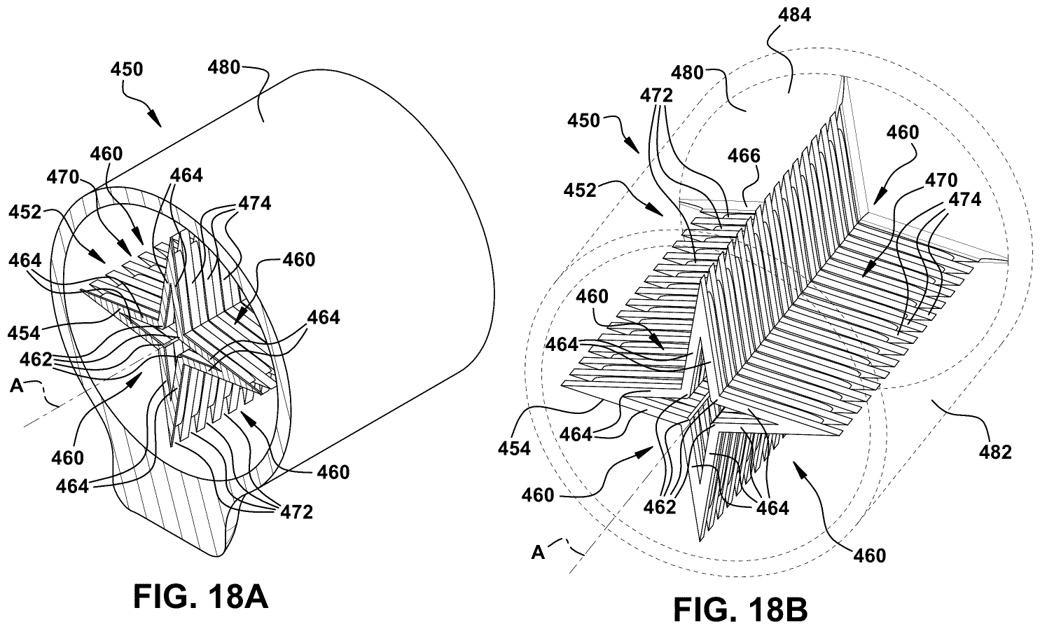

An example gripper 450 is illustrated in FIGS. 18A and 18B and can be similar or identical to the grippers 322, 362 described above in regard to the second FFA assembly 300. The gripper 450 has a stellate gripper tube 452 with a corrugated sidewall 454 that is stellate in cross-section. Specifically, the sidewall 454 is corrugated both axially and radially. Axial corrugations 460 extend the length of the gripper tube 452. Radial corrugations 470 extend about the central axis A and are spaced along the length of the gripper tube 452.

In the example configuration, each axial corrugation 460 includes one inward fold of the sidewall 454. In other words, each axial corrugation includes one of the gripping points 462 and the two adjacent wall sections 464 that converge and meet at that gripping point. The gripper tube 452 of FIGS. 18A and 18B thus includes four longitudinal corrugations 460.

The radial corrugations 470 extend radially about the axis A, transversely of the longitudinal corrugations. The radial corrugations 470 are defined by radially extending grooves 472 that define a series of radially extending ribs 474. The grooves 472 and ribs 474 are spaced alternately along the length of the gripper tube 452.

While four axial corrugation 460, gripper tubes 452 with four gripping points 462 are illustrated in the example configuration, it will be appreciated that the a similar construction can be used to produce gripper tubes having a three or more point stellate configuration. Advantageously, the gripper 450 can be manufactured as a single part using the 3D printing techniques described above.

Opposite end portions 466 of the gripper tube 452, only one of which is shown in the sectional views of FIG. 18B, are connected to an end wall 484 of the gripper housing 480. The sidewall 454 of the gripper tube 452 is spaced from the sidewall 482 of the housing 480, defining the pressurization chamber 486 therebetween. When the pressurization chamber 486 is pressurized with fluid/air, the axial corrugations 460 allow the sidewall 454 to deflect radially inward toward the axis A so that the gripping points 462 can engage and grip the tube. At the same time, with the ends 466 secured to the housing 480, the gripper tube 452 behaves like a cantilevered beam where the accumulating pressure in the chamber yields max deflection towards the center of the “beam,” i.e., the center of the gripper tube 452. As this occurs, the tube material pulls inward on the fixed ends, the radial corrugations 470 provide additional tube flexibility that permits the inward deflection necessary to produce the desired gripping force.

From this, it will be appreciated that the number of axial corrugations in the gripper sidewall, forming its multipoint, stellate shape, determines the degree to which the gripper tube can deflect radially inward. The degree to which the gripper tube can deflect radially inward is inversely proportional to the number of axial corrugations. As the number of axial corrugations grows, the gripper sidewall approaches an inflexible, smooth, circular shape. Thus, while holding tube diameter constant, the radial deflection of a gripper tube having a large number of axial corrugations will be less than that of a gripper tube having fewer axial corrugations.

### Alternative Rotational Actuator

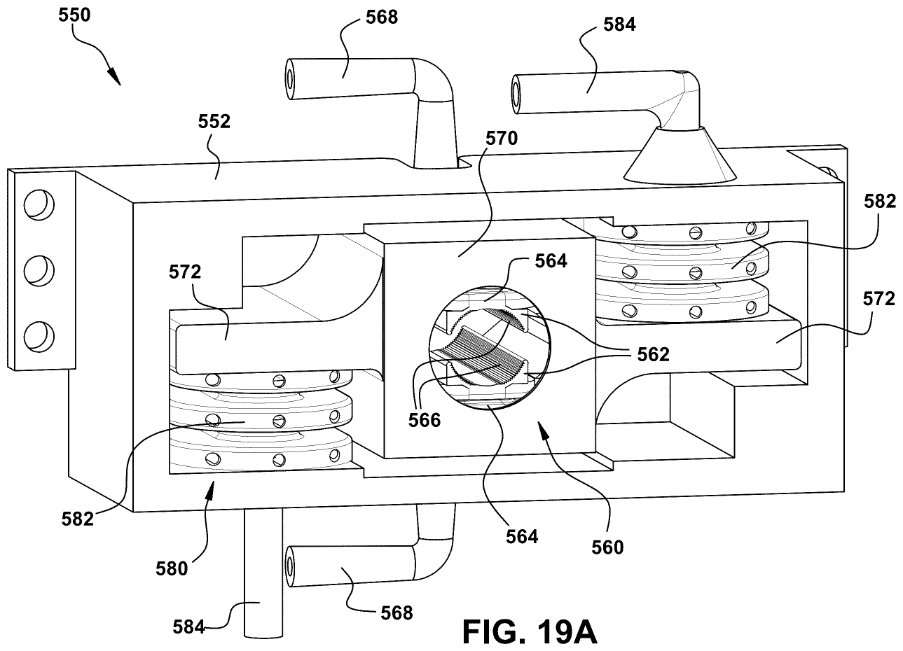

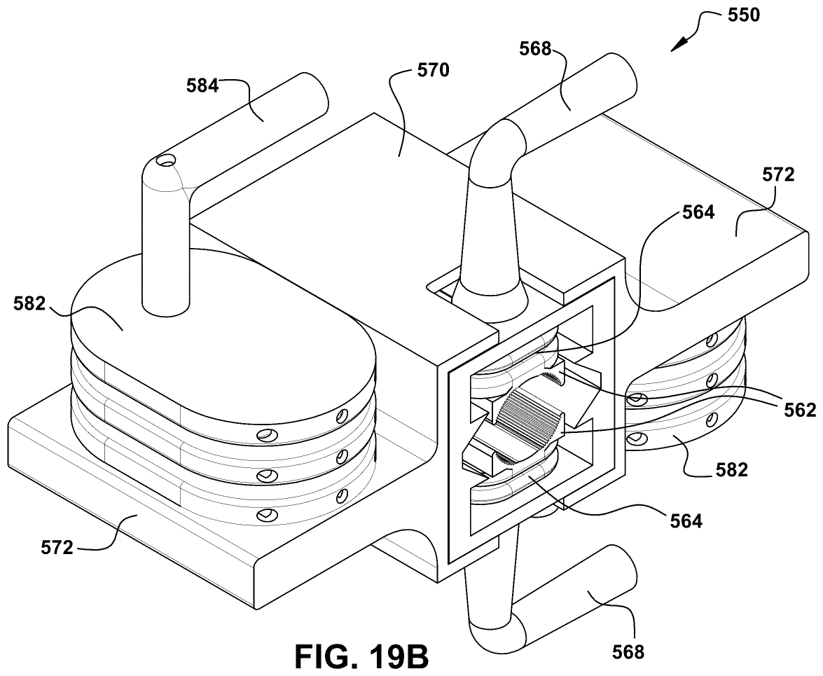

An rotational FFA 550 is shown in FIGS. 19A and 19B. The rotational FFA 550 has a configuration that is similar to that of the rotational FFA 260 of FIGS. 12A-12C. The rotational FFA 550 has a more robust configuration designed to actuate larger tubular elements, such as those that might be encountered with the actuation of the robotic parallelogram arm 20 (see FIG. 1). The rotational FFA 550 thus can have a larger form factor, as it is not located in the limited space adjacent the patient, as is the actuator unit 100 (see again, FIG. 1). Because of this, the components, such as the bellows, can have larger volumes producing larger force exerting areas, yielding greater actuation forces for a given degree of pressurization.

The rotational FFA 550 includes a frame 552 that supports a gripper 560 and a rotational actuator 580. The gripper 560 includes a pair of opposing gripping jaws 562 actuated associated bellows 564 supported in a frame 570. For enhanced gripping, the gripping jaws 562 can include gripping grooves 566. Fluids, such as air, are delivered to the bellows 564 via a fittings 568 to actuate the gripper 560.

The gripper 560 is supported in the frame 552 by the rotational actuator 580, specifically by a pair of bellows 582 that engage a respective arm 572 of the frame 570. Each of the bellows 582 has an associated fitting 584 through which fluids, e.g., air, can be delivered to pressurize and actuate the bellows. When actuated, the bellows 582 rotate the gripper 560 in one direction (CW as viewed in FIGS. 19A and 19B). When pressure is released, the bellows 582 rotate the gripper 560 in the opposite direction (CCW).

### Control Setup

To control the actuation of the concentric tube manipulator 50 using the FFA assemblies 200, 300 of the actuation unit 100, the various grippers, translational actuators, and rotational actuators are charged with air or exhausted to atmospheric pressure, in a specific order, in order to translate or rotate the tubes. Directionality can be achieved by using either the charging or exhausting phase as the actuation state. Throughout actuation, by rule, the tube is griped by a gripper at all times. Thus, the grippers double both as a clutch for connecting/disconnecting power as well as a stabilizing device for when the tube is not being actively actuated.

For example, using the translational FFA to impart tube translation in first direction is achieved by the following sequence:
Charge the translational gripper to grip the tube.
Charge the translational actuator to translate the griped tube in the first direction.
Charge the rotational gripper to grip the tube.
Exhaust the translational gripper to release the tube.
Exhaust the translational actuator to return to the neutral position.

Using the translational FFA to impart tube translation in the opposite direction is achieved by the following sequence:
Charge the rotational gripper to grip the tube.
Exhaust the translational gripper to release the tube.
Charge the translational actuator to move the translational gripper along the tube.
Charge the translational gripper to grip the tube.
Exhaust the rotational gripper to release the tube.
Exhaust the translational actuator to translate the griped tube in the opposite direction.

For example, using the rotational FFA to impart tube rotation in first direction, e.g., CW rotation, is achieved by the following sequence:
Charge the rotational gripper to grip the tube.
Charge the rotational actuator to rotate the griped tube in the CW direction.
Charge the translational gripper to grip the tube.
Exhaust the rotational gripper to release the tube.
Exhaust the rotational actuator to return to the neutral position.

Using the rotational FFA to impart tube rotation in the opposite CCW direction is achieved by the following sequence:
Charge the translational gripper to grip the tube.
Exhaust the rotational gripper to release the tube.
Charge the rotational actuator to rotate the gripper CW about the tube.
Charge the rotational gripper to grip the tube.
Exhaust the translational gripper to release the tube.
Exhaust the rotational actuator to rotate the griped tube in the CCW direction.

The above sequences are executed in rapid succession, one at a time, so that either tube translation or rotation is executed at any given moment, while ensuring that the tube is griped by a gripper at all times. This helps to ensure system safety and maintain accuracy in the axial and rotational tube position measurements obtained by the linear and rotational encoders. The stepwise operation also ensures that the actuation unit cannot impart motion more than one step in the event of a hardware malfunction.

When the charging phase is used as the actuation state, it is referred to as active translation or rotation. Consequently, when the exhausting phase is used as the actuation state, it is referred to as passive translation or rotation. When driving the actuation unit in an open loop manner, the orifice area of the control valve is maximized, leading to full step sizes at every actuation state. This is called full-step control mode.

The other operation mode, sub-step control, is used when the difference between the current and target position is less than one step size. This mode drives the actuation unit in a closed loop manner continuously adjusting the valve orifice area during the actuation state using a proportional spool valve. This mode uses closed loop control and implements a robust, boundary layer sliding mode controller to handle the nonlinear dynamics of the system. This precise sub-step control enables the translation and rotation of the needle to be keyed according to the needle's helical pitch. In the LITT epilepsy treatment implementation described above, concentric tube actuation using the sub-step control allows the system to avoid the shearing of brain tissue.

From the above description of the invention, those skilled in the art will perceive improvements, changes and modifications. Such improvements, changes and modifications within the skill of the art are intended to be covered by the appended claims.

1. A flexible fluidic actuator for robotically imparting translational or rotational motion to a tube of a concentric tube manipulator, the flexible fluidic actuator comprising:
a gripper that is actuatable in response to fluid pressure to grasp the tube, and de-actuatable in response to relieving fluid pressure to release the tube; and
one or more actuator bellows that are actuatable in response to fluid pressure to move the gripper to an actuated position, and de-actuatable in response to releasing fluid pressure to return the gripper to a de-actuated position, wherein the gripper is configured to move to the actuated position in a first translational or rotational direction, and to move to the de-actuated position in a second translational or rotational direction, opposite the first translational or rotational direction;
wherein the one or more actuator bellows are arranged on an actuator frame and configured to support the gripper, and wherein the flexible fluidic actuator is configured so that the frame, actuator bellows, and gripper occupy the same axial length.

2. The flexible fluidic actuator recited in claim 1, wherein the flexible fluidic actuator is configured to impart the translational or rotational motion to the tube in a first direction by sequentially:
actuating the gripper to grasp the tube,
actuating the one or more actuator bellows to move the gripper to the actuated position thereby imparting translational or rotational movement to the grasped tube in the first direction,
de-actuating the gripper to release the tube, and
de-actuating the one or more actuator bellows to return the gripper to the de-actuated position;
wherein the flexible fluidic actuator is configured to impart the translational or rotational motion to the tube in a second direction, opposite the first direction by sequentially:
de-actuating the gripper,
actuating the one or more actuator bellows to move the gripper to the actuated position,
actuating the gripper to grasp the tube,
de-actuating one or more actuator bellows to move the gripper to the de-actuated position thereby imparting translational or rotational movement to the grasped tube in the second direction.

3. The flexible fluidic actuator recited in claim 2, wherein the gripper comprises a pair of opposing gripper bellows supported on a gripper frame and configured to move a pair of opposing gripper jaws toward each other in the actuated condition of the gripper and to move the gripper jaws away from each other in the de-actuated state of the gripper.

4. The flexible fluidic actuator recited in claim 3, wherein the flexible fluidic actuator is a rotational flexible fluidic actuator configured to rotate the tube in opposite directions about the tube axis, and wherein the one or more actuator bellows comprise a pair of actuator bellows configured to move opposite lateral ends of the gripper frame in opposite directions in order to impart rotational movement to the gripper.

5. The flexible fluidic actuator recited in claim 3, wherein the flexible fluidic actuator is a translational flexible fluidic actuator configured to translate the tube in opposite directions along the tube axis, and wherein the one or more actuator bellows comprises a peripheral bellow that extends along a peripheral portion of the actuator frame and defines an open central window, the gripper being connected to the peripheral bellow and positioned in the central window, wherein the gripper is configured to move axially with the peripheral bellow in opposite directions in order to impart translational movement to the gripper.

6. The flexible fluidic actuator recited in claim 2, wherein the gripper comprises a gripper tube supported within a gripper housing, the gripper tube comprising a sidewall that defines a longitudinally extending central opening configured to receive the tube, the gripper tube sidewall having a stellate corrugated configuration;
wherein the gripper housing surrounds the gripper tube, defining a pressure chamber that extends circumferentially around the gripper tube between the gripper housing and the gripper tube;
wherein the pressure chamber is pressurized with fluid in the actuated condition of the gripper, causing the gripper tube sidewall to deflect radially inward under fluid pressure into engagement with the tube to grasp the tube;
wherein fluid pressure in the pressure chamber is released in the deactuated condition of the gripper, which causes the gripper tube sidewall to expand radially and release the tube.

7. The flexible fluidic actuator recited in claim 6, wherein the corrugated configuration of the gripper tube sidewall comprises axial corrugations and radial corrugations, wherein the axial corrugations extend the length of the gripper tube sidewall and are spaced about the circumference of the gripper tube sidewall, and wherein the radial corrugations extend about a central axis of the gripper tube and are spaced along the length of the gripper tube.

8. The flexible fluidic actuator recited in claim 7, wherein the gripper tube is supported at each end by the gripper housing, wherein the radial corrugations are configured to cause the gripper tube sidewall to behave like a cantilevered beam where the accumulating pressure in the chamber yields max deflection towards the center of the gripper tube.

9. The actuation unit recited in claim 6, wherein the stellate configuration of the gripper tube comprises a four-point stellate configuration defining four corrugations that produce four points of contact with the tube in the actuated condition of the gripper.

10. The actuation unit recited in claim 6, wherein the stellate configuration of the gripper tube defines peaks positioned proximally to the gripper housing and valleys positioned proximate to the tube.

11. The flexible fluidic actuator recited in claim 6, wherein the flexible fluidic actuator is a rotational flexible fluidic actuator configured to rotate the tube in opposite directions about the tube axis, and wherein the one or more actuator bellows comprise a pair of actuator bellows configured to move opposite lateral ends of the gripper housing in opposite directions in order to impart rotational movement to the gripper.

12. The flexible fluidic actuator recited in claim 6, wherein the flexible fluidic actuator is a translational flexible fluidic actuator configured to translate the tube in opposite directions along the tube axis, and wherein the one or more actuator bellows comprises a ring-shaped cylindrical bellow that defines an open cavity configured to receive and support the gripper, wherein the gripper is configured to move axially with the cylindrical bellow in opposite directions in order to impart translational movement to the gripper.

13. The flexible fluidic actuator recited in claim 1, wherein the gripper and the one or more actuator bellows are configured for pneumatic actuation.

14. An actuator unit for actuating inner and outer tubes of a concentric tube manipulator, comprising:
a first flexible fluidic actuator according to claim 1, wherein the first flexible fluidic actuator is configured to robotically impart translational motion to the inner tube;
a second flexible fluidic actuator according to claim 1, wherein the second flexible fluidic actuator is configured to robotically impart rotational motion to the inner tube;
a third flexible fluidic actuator according to claim 1, wherein the third flexible fluidic actuator is configured to robotically impart translational motion to the outer tube; and
a fourth flexible fluidic actuator according to claim 1, wherein the fourth flexible fluidic actuator is configured to robotically impart rotational motion to the outer tube.

15. The actuator unit recited in claim 14, further comprising a frame configured to support the first, second, third, and fourth flexible fluidic actuators, wherein the third and fourth flexile fluidic actuators are fixed to the frame, wherein the first and second flexible fluidic actuators are configured to move axially on the frame in response to translational movement of the outer tube with respect to the frame.

16. The actuator unit recited in claim 15, wherein the first and second flexible fluidic actuators are supported by a first carriage comprising rollers configured to roll along the frame to allow the first and second flexible fluidic actuators to move axially on the frame.

17. The actuator unit recited in claim 16, further comprising:
a second carriage comprising rollers configured to roll along the frame in response to translational movement of the inner tube with respect to the frame;
wherein the first carriage comprises a linear encoder configured to measure the axial position of the outer tube, and a rotational encoder configured to measure the rotational position of the outer tube; and
wherein the second carriage comprises a linear encoder configured to measure the axial position of the inner tube, and a rotational encoder configured to measure the rotational position of the inner tube.

18. The actuator unit recited in claim 16, wherein all of the components of the actuator unit are constructed using an MRI compatible material.

19. A surgical robotic system for performing a robotic intervention inside an MRI tube, comprising the actuator unit recited in claim 15, a robotic parallelogram arm to support the actuator unit in the MRI tube and to robotically control the position of the actuator unit in the MRI tube, and a positioning platform configured to control the position of the robotic parallelogram arm and to fix the robotic parallelogram arm to a patient bed in the MRI tube.

20. The surgical robotic system recited in claim 19, wherein the robotic parallelogram arm comprises a pair of flexible fluidic actuator each configured to control a degree of freedom of the parallelogram arm.
